(19) United States
(12) Patent Application Publication
(10) Pub. No.: US 2024/0027328 A1
(43) Pub. Date: Jan. 25, 2024

(54) METHOD FOR PREDICTING GENERATED AMOUNT OF SILICA SCALE

(71) Applicant: FUJI ELECTRIC CO., LTD., Kawasaki-shi (JP)

(72) Inventors: Tianlong JIANG, Hachioji-city (JP); Takayuki HIROSE, Sagamihara-city (JP); Azusa WADA, Koganei-city (JP); Shinya UI, Hachioji-city (JP)

(73) Assignee: FUJI ELECTRIC CO., LTD., Kawasaki-shi (JP)

(21) Appl. No.: 18/476,086

(22) Filed: Sep. 27, 2023

Related U.S. Application Data
(63) Continuation of application No. PCT/JP2022/039764, filed on Oct. 25, 2022.

(30) Foreign Application Priority Data
Oct. 25, 2021 (JP) 2021-174075

(51) Int. Cl. G01N 17/00; G16C 60/00; F24T 50/00

(52) U.S. Cl. CPC G01N 17/008; G16C 60/00; F24T 50/00

(57) ABSTRACT

The generated amount of silica scale under complicated conditions is accurately predicted. A method for predicting a generated amount of silica scale includes: a step of acquiring a temperature at a prediction portion at which the adherence of silica scale needs to be predicted, Tₛ (K), and/or time until fluid containing silicic acid reaches the prediction portion, tₛ (min), and a step of calculating the amount of silica adhered at the prediction portion based on the predictive equation of the saturation concentration of silica depending on the temperature and/or the predictive curve of the concentration of silica dissolved depending on the time, wherein the predictive equation of the saturation concentration of silica and the predictive curve of the concentration of silica dissolved are obtained based on k₁, k₂, k_{B}, and kₐ in a three-step precipitation equilibrium reaction model represented by the following

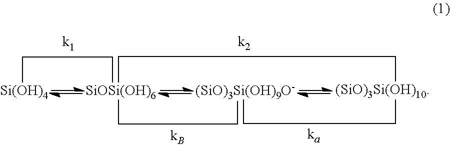

### TECHNICAL FIELD

The present invention relates to a method for predicting the generated amount of silica scale, to a prediction system, and to a geothermal power generation system comprising the prediction system. The present invention relates particularly to a prediction method and to a prediction system that enables prediction of the amount of silica scale that will be generated by simulation, without collecting data by experiment, and to a geothermal power generation system comprising the prediction system.

### BACKGROUND ART

In a plant system such as a geothermal power generation system using groundwater, silica scale derived from silica dissolved in water adheres to equipment and piping constituting the plant system. Silica scale is a strong deposit mainly constituted of a polymer of Si and O. A decrease in reduction capability due to silica scale generation, a decrease in power generation efficiency, or the like is problematic, especially in geothermal power generation plants.

Various methods for predicting the amount of scale generated from the properties and conditions of hot water have been proposed.

The kinetics of the reaction of silicic acid and the surface of amorphous silica in an aqueous NaCl solution is known (for example, refer to Non-Patent Document 1).

A method for predicting scale growth involving predicting the thickness of scale to be generated in a pit from the speed of fluid in the pit is known (for example, refer to Patent Document 1). A method for quantifying and evaluating scale involving weighing a device that can be locally heated to measure the mass of scale from the comparison between the masses before and after heating is known (for example, refers to Patent Document 2). A method for assuming the scale thickness based on the temperature of fluid flowing through piping or the outer surface of the piping, the thermal conductivity of the scale, and the like is known (for example, refer to Patent Document 3). A method for suppressing scale involving spraying a scale suppressant depending on the scale components contained in vapor is known (for example, refer to Patent Document 4).

### REFERENCE DOCUMENT LIST

### Patent Documents

Patent Document 1: Japanese Patent Application, Laid-Open No. 2002-257030 A
Patent Document 2: Japanese Patent Application, Laid-Open No. 2019-027961 A
Patent Document 3: International Publication WO 2019/202981 A1
Patent Document 4: Japanese Patent Application, Laid-Open No. 2020-12456 A

### Non-Patent Document

Non-Patent Document 1: Journal of Colloid and Interface Science, Volume 110, Issue 1, March 1986, Pages 40-46

### SUMMARY OF THE INVENTION

### Problem to be Solved by the Invention

However, the method disclosed in Non-Patent Document 1 is an empirical technique, and it requires a large number of measured values to establish a predictive equation, and experimental conditions cannot be further accurately controlled. Accurate predicted values are therefore not obtained. Every time the conditions vary, the empirical predictive equation needs to be modified by repeating the measurement, and the method is therefore lacking in versatility.

In the methods disclosed in Patent Documents 1 to 4, empirical values such as a large amount of experimental data and observed data were required to establish a predictive equation for predicting the generated amount of silica scale. The use of a special apparatus or operations such as stopping a plant system such as a geothermal power generation system to disassemble a part of the apparatus are required for acquiring data. Furthermore, the predictive equation established by the empirical method is easily affected by variation of conditions. Enormous amounts of data are necessary to obtain an accurate predictive equation.

In conventional technology, it was thus difficult to accurately predict the generated amount of silica scale. For example, after plant systems such as geothermal power generation systems stop unexpectedly, and the powder generating capacity decreases, maintenance must therefore be conducted. It was therefore disadvantageous to reduce sales due to a decrease in the amount of power sold. A problem was that when the maintenance was performed in advance to avoid a decrease in power generating capacity and unexpected stoppage, the amount of the silica scale generated was not able to be accurately understood, the maintenance period therefore could not be optimized, and the maintenance cost increased due to an increase in the number of times maintenance had to be conducted.

### Means for Solving the Problem

The present inventors have examined the quantification of the generated amount of silica scale by simulation independently of experimentation. As a result, the present inventors have conceived a reaction model of a silica polymerization reaction in hot water, established a technique for calculating various parameters necessary for predicting the saturation concentration of silica and the concentration of silica dissolved, and completed the present invention.

The problem to be solved by the present invention can be solved by the following preferable aspects.

[1] A method for predicting a generated amount of silica scale, including:
a step of acquiring a temperature at a prediction portion at which adherence of silica scale needs to be predicted, Tₛ (K), and/or time until fluid containing silicic acid reaches the prediction portion, tₛ (min), and
a step of calculating an amount of silica adhered at the prediction portion based on a predictive equation of a saturation concentration of silica depending on the temperature and/or a predictive curve of a concentration of silica dissolved depending on the time,
in which the predictive equation of the saturation concentration of silica and the predictive curve of the concentration of silica dissolved are obtained based on k₁, k₂, k_{B}, and kₐ in a three-step precipitation equilibrium reaction model represented by the following Formula (1):

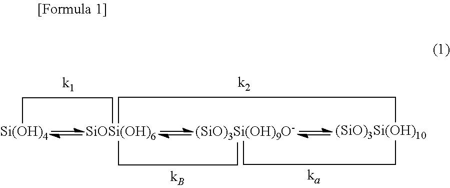

in which
k₁ is a reaction equilibrium constant between Si(OH)₄ and SiOSi(OH)₆,
k₂ is a reaction equilibrium constant between SiOSi(OH)₆ and (SiO)₃OSi(OH)₁₀,
k_{B} is an ionization equilibrium constant between SiOSi(OH)₆ and (SiO)₃Si(OH)₉O⁻, and
kₐ is a silica acid dissociation constant between (SiO)₃Si(OH)₉O⁻ and (SiO)₃OSi(OH)₁₀.
   [2] The method according to [1], in which the silica acid dissociation constant kₐ is obtained by quantum chemical calculation and linear fitting correction based on free energy change ΔG in equilibrium reaction between (SiO)₃Si(OH)₉O⁻ and (SiO)₃OSi(OH)₁₀.
   [3] The method according to [2], in which relationship between the silica acid dissociation constant kₐ and the free energy change ΔG is represented by the following equation:

pkₐ=pΔG+q

in which p and q are constants.
[4] The method according to [3], in which p is 0.19 to 0.24, and q is −56 to −51.
[5] The method according to [1], in which the predictive curve of the concentration of silica dissolved C is obtained by fitting plotting of concentrations of silica dissolved at two or more different time points obtained by first-principle calculation based on an initial concentration of silica, Cᵢ, and Formula (1),
a frequency factor to be used for correcting the fitting in an initial stage of the reaction, A, is represented by the following equation:

A=m[exp(nT)] (3)

in which m and n are constants, and are calculated based on k₁, k₂, k_{B}, and kₐ.
[6] The method according to [5], in which m is 2.0 to 3.1, and n is 0.083 to 0.085.
[7] The method according to [1], in which the predictive equation of the saturation concentration of silica Ce is represented by a saturation concentration of silica at a temperature T, Ce₁:

Ce₁=a₁[exp(b₁T)] (2)

in which
a₁ and b₁ are constants calculated based on k₁, k₂, k_{B}, and kₐ, and T represents polymerization reaction temperature.
   [8] The method according to [7], in which a₁ is 18 to 32, and b₁ is 0.005 to 0.010.
   [9] The method according to [1], in which a predictive equation of the saturation concentration of silica Ce is represented by a saturation concentration of silica at a temperature T and a pH of 0 or more and less than 7, Ce₂:

Ce₂=R{a₂[exp(b₂T)]} (4)

in which
a₂ and b₂ are constants calculated based on k₁, k₂, k_{B}, and kₐ,
R is an effective activity coefficient calculated based on the pH, and
T represents polymerization reaction temperature.
   [10] The method according to [9], in which a calculation equation of the effective activity coefficient R is represented by the following equation:

−logR=A_{R}Z²{E/(1+B_{R}cE)} (5)

in which

A_{R}=1.825*10⁶(εT)^{-3/2},

B_{R}=50.3*(εT)^{-1/2},
a charge number, Z, is a constant selected from 1 or 2, and an effective diameter coefficient, c, is 4,
E is an effective ionic strength represented by the following Equation (6):

E={I+(hydrogen ion concentration)}/[1+B_{R}c[I+(hydrogen ion concentration)] (6)

in which I is a solute ionic strength.
[11] The method according to [9], in which a₂ is 16 to 36, and b₂ is 0.003 to 0.015.
[12] The method according to [1], in which a predictive equation of the saturation concentration of silica Ce₃ is represented by a saturation concentration of silica at a temperature T and a pH of more than 7 and 14 or less, Ce₃:

Ce₃=(1−J){a₃[exp(b₃T)]} (7)

in which
a₃ and b₃ are constants calculated based on k₁, k₂, k_{B}, and kₐ,
J is an effective reaction factor calculated based on abundance fractions of a silica monomer ion and a silica dimer ion, and
T represents polymerization reaction temperature.
   [13] The method according to [12], in which a calculation equation of the effective reaction factor J is represented by the following equation:

J=(X−Xi₁−Xi₂)/X (8)

in which
X is a total amount of silica,
Xi₁ is the abundance fraction of the silica monomer ion calculated from an acid dissociation constant, kₐⱼ, and
Xi₂ is the abundance fraction of the silica dimer ion calculated from the acid dissociation constant kₐⱼ.
   [14] The method according to [12, in which a₃ is 6 to 34, and b₃ is 0.005 to 0.015.
   [15] The method according to [1], in which the amount of silica adhered is predicted by
a step of acquiring a total concentration of silica in the fluid containing silicic acid, Cₜ, and
a step of calculating the amount of silica adhered based on the total concentration of silica Cₜ and the saturation concentration of silica.
   [16] The method according to [1], in which the amount of silica adhered is predicted by a step of calculating the amount of silica adhered based on the predictive curve of the concentration of silica dissolved.
   [17] A system for predicting a generated amount of silica scale, including:
a device that acquires a temperature at a prediction portion at which adherence of silica scale needs to be predicted, Tₛ (K), and/or time until fluid containing silicic acid reaches the prediction portion, tₛ (min.), and
a device that calculates an amount of silica adhered at the prediction portion based on a predictive equation of a saturation concentration of silica depending on the temperature and/or a predictive curve of a concentration of silica dissolved depending on time,
in which the predictive equation of the saturation concentration of silica and the predictive curve of the concentration of silica dissolved are obtained based on k₁, k₂, k_{B}, and kₐ in a three-step precipitation equilibrium reaction model represented by the following Formula (1):

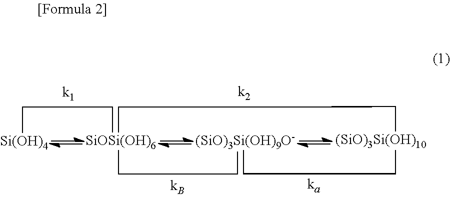

in which
k₁ is a reaction equilibrium constant between Si(OH)₄ and SiOSi(OH)₆,
k₂ is a reaction equilibrium constant between SiOSi(OH)₆ and (SiO)₃OSi(OH)₁₀,
k_{B} is an ionization equilibrium constant between SiOSi(OH)₆ and (SiO)₃Si(OH)₉O⁻, and
kₐ is a silica acid dissociation constant between (SiO)₃Si(OH)₉O⁻ and (SiO)₃OSi(OH)₁₀.
   [18] A geothermal power generation system, including:
a gas-liquid separator that separates geothermal fluid drawn from a production well into a gas component and a liquid component;
a turbine that is disposed downstream of the gas-liquid separator and that is configured to be rotatable by the gas component separated in the gas-liquid separator;
piping that delivers the liquid component separated in the gas-liquid separator to an injection well; and
the system for predicting a generated amount of silica scale according to [17].

### Effects of the Invention

According to a method for predicting the generated amount of silica scale in accordance with the present invention, the generated amount of silica scale can be predicted independently of empirical values such as experimental values or observed values easily and accurately even under complicated conditions. Cost due to stoppages of plant systems or an increase in the number of times maintenance is conducted can be minimized, and the plant systems can be steadily and efficiently managed thereby. Moreover, the method is also effective in the design of plant systems in which silica scale may be generated.

### BRIEF DESCRIPTION OF THE DRAWINGS

FIG. 1 is a flow chart for calculating the saturation concentration of silica Ce₁ and the concentration of silica dissolved C₁ in a prediction method according to a first aspect of the present invention.

FIG. 2 is a graph showing an example of a predictive curve showing the relationship between the polymerization reaction temperature T and the saturation concentration Ce₁ at a pH of 7 that can be used in the prediction method according to the first aspect of the present invention.

FIG. 3 is a graph showing an example of predictive curves showing the relationship among the polymerization reaction time t, the concentration of silica dissolved C₁, and the amount of silica deposited at 100° C. and a pH of 7 that can be used in the prediction method according to the first aspect of the present invention.

FIG. 4 is a semilogarithmic graph showing a predictive curve of the frequency factor A used in the fitting for predicting the dissolved concentration in the initial stage of the reaction accurately, and the vertical axis is a logarithm scale. Experimental values at a time point of 5 min and temperatures can be reproduced using the frequency factor A.

FIG. 5 is a flow chart for calculating the saturation concentration of silica Ce₂ and the concentration of silica dissolved C₂ in a prediction method according to a second aspect of the present invention.

FIG. 6 is a graph showing an example of predictive curves showing the relationships between the polymerization reaction temperature T and the saturation concentration Ce₂ at pHs of 5.5 and 7.0 that can be used in the prediction method according to the second aspect of the present invention.

FIG. 7 is a graph showing an example of predictive curves showing the relationship among the polymerization reaction time t, the concentration of silica dissolved C₂, and the amount of silica deposited at 150° C. and a pH of 5.5 that can be used in the prediction method according to the second aspect of the present invention.

FIG. 8 is a flow chart for calculating the saturation concentration of silica Ce₃ and the concentration of silica dissolved C₃ in a prediction method according to a third aspect of the present invention.

FIG. 9 is a graph showing a change in the effective reaction factor J in the region of 7 to 14.

FIG. 10 is a graph showing an example of predictive curves showing the relationships between the polymerization reaction temperature T and the saturation concentration Ce₃ at pHs of 5.5, 7.0, and 9.0 that can be used in the prediction method according to the third aspect of the present invention.

FIG. 11 is a graph showing an example of predictive curves showing the relationship among the polymerization reaction time t, the concentration of silica dissolved C₃, and the amount of silica deposited at 100° C. and a pH of 9.0 that can be used in the prediction method according to the third aspect of the present invention.

FIG. 12 is a conceptual diagram illustrating a geothermal power generation system including a system for predicting the generated amount of silica scale according to one embodiment of the present invention.

### MODE FOR CARRYING OUT THE INVENTION

Hereinafter, embodiments of the present invention will be described with reference to the drawings. However, the present invention is not limited to the embodiments described below.

### First Embodiment: Method for Predicting Generated Amount of Silica Scale

According to a first embodiment, the present invention relates to a method for predicting the generated amount of silica scale. The method for predicting the generated amount of silica scale includes the following steps:
a step of acquiring a temperature at a prediction portion at which the adherence of silica scale needs to be predicted, Tₛ (K), and/or time until fluid containing silicic acid reaches the prediction portion, tₛ (min), and
a step of calculating the amount of silica adhered at the prediction portion based on a predictive equation of the saturation concentration of silica depending on the temperature and/or a predictive curve of the concentration of silica dissolved depending on the time.

The present embodiment relates more specifically to a method for predicting the generated amount of silica scale in a plant system in which fluid containing silicic acid is circulated. The fluid containing silicic acid refers to fluid containing Si and OH, including Si(OH)₄, Si(OH)₃O⁻, SiO₂(OH)₂²⁻, Si₂O₂(OH)₅⁻, and/or Si₂O₃(OH)₄²⁻, but is not limited to these. The plant system may be a plant system including piping and equipment in which the fluid containing silicic acid is circulated, and is, for example, a plant system in which the adherence of silica scale can lead to stoppage or problems in the plant system. Examples of the plant system include, but are not limited to, a geothermal power generation system, a boiler system, a system including cooling water piping, and a water treatment system.

The fluid containing silicic acid may be, for example, water that can contain silicic acid, and examples thereof include, but are not limited to, groundwater, tap water, geothermal water, or wastewater derived from these. For example, if the plant system is a geothermal power generation system, the fluid containing the silicic acid may be geothermal water. The fluid is not limited to a liquid, and it also includes a mixture of water and gas such as vapor.

In the present embodiment, three predictive equations were established with respect to the saturation concentration of silica depending on the temperature. Methods for deriving the predictive curves of the concentrations of silica dissolved depending on time were established based on these predictive equations, respectively. Hereinafter, aspects will be described.

### First Aspect

In a first aspect of the method according to the present embodiment, the generated amount of silica scale at the any portion of a plant system is predicted based on the predictive equation of the saturation concentration of silica Ce₁ depending on the temperature T. In the first aspect, a predictive equation in the case of pH 7 is provided. The saturation concentration of silica Ce₁ that the predictive equation yields refers to the weight percent concentration (unit: ppm) of a solution in which silica that can be generated by the condensation polymerization reaction of silicic acid described above (containing monomers and various silica polymers including dimers or greater) is dissolved, and the solution reaches a saturated solution at a predetermined temperature T. In other words, the saturation concentration of silica Ce₁ refers to the maximum concentration at which silica (containing monomers and various silica polymers including dimers or higher) can be dissolved at the predetermined temperature T. Silica scale refers to silica that cannot be dissolved in fluid, and is deposited. Although the deposited silica can usually be a tetramer or a polymer with a higher polymerization degree than the tetramer, the polymerization degree of the polymer is not particularly limited.

The present inventors have established a three-step precipitation equilibrium reaction model represented by the following Formula (1) to obtain the predictive equation of the saturation concentration of silica Ce₁.

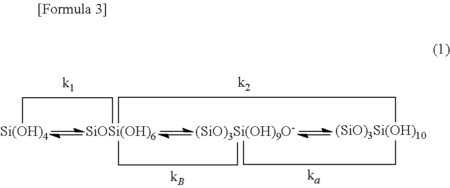

wherein, in Formula (1),
k₁ is a reaction equilibrium constant between Si(OH)₄ and SiOSi(OH)₆,
k₂ is a reaction equilibrium constant between SiOSi(OH)₆ and (SiO)₃OSi(OH)₁₀,
k_{B} is an ionization equilibrium constant between SiOSi(OH)₆ and (SiO)₃Si(OH)₉O⁻, and
kₐ is a silica acid dissociation constant between (SiO)₃Si(OH)₉O⁻ and (SiO)₃OSi(OH)₁₀.

The silica polymerization reaction has been calculated based on a two-step model having reversible reaction that generates SiOSi(OH)₆ from Si(OH)₄ and irreversible reaction that generates (SiO)₃OSi(OH)₁₀ from SiOSi(OH)₆ until now. In the present invention, the adoption of the model of the above-mentioned Formula (1) further in view of the precipitation equilibrium reaction instead of this two-step model enables prediction including the kinetics of a chemical species represented by (SiO)₃Si(OH)₉O⁻ that has not been considered until now.

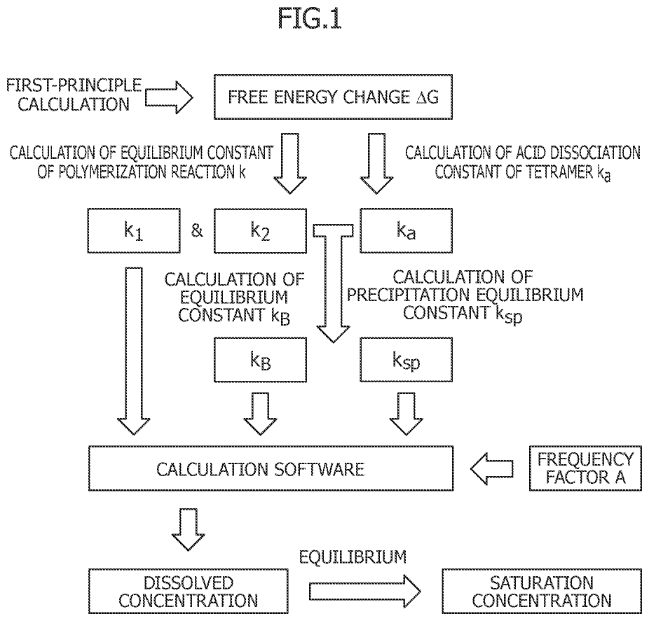

Then, the derivation of the predictive equation of the saturation concentration of silica Ce₁ and the curve of the concentration of silica dissolved C₁ will be described. FIG. 1 is a flow chart for calculating the saturation concentration of silica Ce₁ and the concentration of silica dissolved C₁.

In the present technique, free energy changes ΔG in the reactions in the steps of Formula (1) are first obtained from the free energy of fluid (for example, water) that dissolves four chemical species represented by Formula (1) and silicic acid. The k₁ and k₂ are obtained from the values of the free energy changes ΔG. The temperature T in the calculation equation of the equilibrium constants of the polymerization reaction is polymerization reaction temperature. As the acid dissociation constant kₐ, the following value calculated from the free energy change ΔG by the quantum chemical calculation and linear fitting correction can be used.

pkₐ=pΔG+q

In the equation, p and q are constants, and ΔG is the value of the free energy change in the equilibrium reaction between (SiO)₃Si(OH)₉O⁻ and (SiO)₃OSi(OH)₁₀. More specifically, p may be 0.19 to 0.24, and q may be −56 to −51. Preferably, p may be 0.21 to 0.22, and q may be −54 to −52.

The relationship between the polymerization reaction temperature T and the saturation concentration of silica Ce₁ (ppm) is calculated as the following Equation (2) by these calculations.

Ce₁=a₁[exp(b₁T)] (2)

In Equation (2), a₁ and b₁ are constants, and are values obtained by the calculation described in the above-mentioned flow chart. T (K) represents polymerization reaction temperature. The temperature range of T may be about 250 K to 500 K. More specifically, a₁ may be 18 to 32, and b₁ may be 0.005 to 0.010. Preferably, a₁ may be 20 to 30, and b₁ may be 0.006 to 0.009.

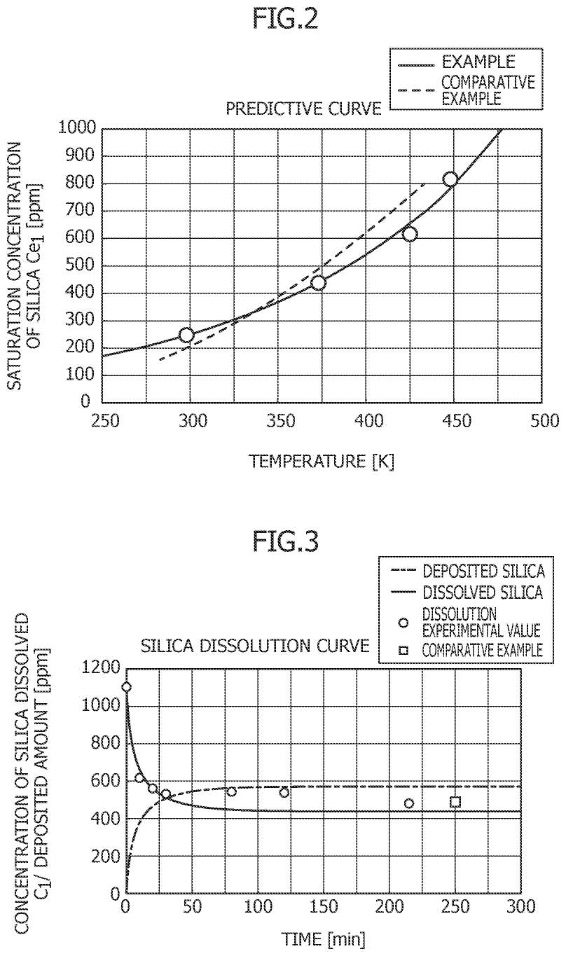

FIG. 2 is an example of Equation (2) showing the predictive curve of the saturation concentration of silica Ce₁. In FIG. 2, the predictive equation according to the present invention is shown by a solid line. The dependence of the saturation concentration of silica Ce₁ on the temperature T can be computed by this predictive equation. The temperature T is polymerization reaction temperature (unit: K) in the three-step precipitation equilibrium reaction model represented by Formula (1). When the amount of silica scale adhered is predicted in the prediction method according to the present embodiment, the temperature Tₛ at a predetermined prediction portion at which the amount of scale adhered in the actual plant system needs to be predicted is used as the polymerization temperature, and can be used for predictive calculation. For example, the temperature Tₛ at the prediction portion may be the temperature of the fluid that can contain silica at the prediction portion of the plant system.

A specific method for predicting the amount of silica scale adhered based on Equation (2) will be described. The prediction method includes the following steps a) to d):
a) a step of acquiring the temperature at a prediction portion at which the adherence of silica scale needs to be predicted, Tₛ (K),
b) a step of calculating the saturation concentration of silica Ce₁ at the prediction portion from the temperature Tₛ and the predictive Equation (2),
c) a step of acquiring the total concentration of silica in fluid containing silicic acid Cₜ, and
d) a step of calculating the amount of silica adhered based on the total concentration of silica Cₜ and the saturation concentration of silica Ce₁.

In the step a), the prediction portion at which the adherence of silica scale needs to be predicted is a portion with which fluid containing silicic acid contacts in a plant system in which the prediction method according to the present embodiment is performed, and is a portion to which silica scale may adhere. For example, in the geothermal power generation system, the portion may be a turbine member such as a turbine blade or a rotor; piping; a heat exchanger; or the like, but the portion is not limited to these. Although the method for acquiring the temperature Tₛ (K) is not particularly limited, for example, the temperature of fluid or equipment at the prediction portion is observed with a device for measuring temperature such as a temperature sensor installed in a specific place, the fluid temperature is computed from the observed temperature of the equipment as necessary, and can be defined as Tₛ. Alternatively, the temperature of the fluid or the equipment at the prediction portion is computed by simulation and the like in view of the operating conditions of the plant system, the fluid temperature is computed from the observed temperature of the equipment as necessary, and can also be defined as Tₛ.

The step b) can be implemented with an arithmetic unit that can perform calculation according to the predictive Equation (2). For example, the arithmetic unit may be a computer in which a specific arithmetic program is incorporated, but it is not particularly limited.

In the step c), the total concentration of silica Cₜ is the total concentration of silica present in fluid fed to the plant system in which the present invention is implemented. In the calculation, the weight percent concentration (ppm) of a silica monomer (Si(OH)₄) in the fluid calculated based on the amount of all the Si atoms present in the fluid is defined as the total concentration of silica Cₜ. It is believed that silica in an amount obtained by deducting the saturation concentration of silica Cₑ from this are deposited and adhered as silica scale. For example, in the geothermal power generation system, the total concentration of silica Cₜ can be calculated as follows. Geothermal water drawn from a production well is first analyzed to obtain the mass or molar amount of Si atoms in the geothermal water. The weight percent concentration of the silica monomers in the geothermal water is then found by calculation on the assumption that all the Si atoms form silica monomers (Si(OH)₄). In the method according to the present embodiment, it is presumed that a part obtained by deducting the saturation concentration from the total concentration of silica Cₜ is deposited as silica scale.

In the step d), a difference between the total concentration of silica Cₜ obtained by the step c) and the saturation concentration of silica Ce₁ can be calculated by an arithmetic unit to obtain the amount of silica scale adhered. The arithmetic unit may be a computer in which a specific arithmetic program is incorporated in the same way as in the step b), but it is not particularly limited.

In the first aspect, the generated amount of silica scale at any portion of the plant system is also then predicted based on the predictive curve of the concentration of silica dissolved C₁ depending on the time. In the prediction method, the predictive curve showing the relationship between the time t and the concentration of silica dissolved C₁ are obtained by calculation, and the amount of silica adhered is calculated from the predictive curve.

The concentration of silica dissolved C₁ is the concentration of the silica dissolved in the fluid at the time t (min) when the time point of the polymerization start is defined as zero. The concentration of silica used here is the weight percent concentration (ppm) of the silica monomer (Si(OH)₄) in the fluid calculated based on the amount of Si atoms dissolved in the fluid in the same way as in the above-mentioned description of the total concentration of silica Cₜ. The initial concentration of silica Cᵢ is the concentration of silica dissolved in the fluid at the time point of the polymerization start (t=0). The initial concentration of silica Cᵢ is also expressed as the weight percent concentration (ppm) of the silica monomer (Si(OH)₄) in the fluid calculated based on the amount of Si atoms dissolved in the fluid. In the three-step precipitation equilibrium reaction model represented by Formula (1), the time t is time (min) when the time point when Si(OH)₄ is dissolved in the fluid is defined as zero. In the prediction method according to the present aspect, the time point recognized as the time point of the polymerization reaction start in the actual plant system is defined as zero, and can be used for the predictive calculation. For example, in the geothermal power generation system, the time point when geothermal water is drawn from a production well can be defined as zero.

The method for acquiring the predictive curve showing the relationship between the time t and the concentration of silica dissolved C₁ will first be described. The method for acquiring the predictive curve includes the following steps i) to iii).
i) a step of acquiring the initial concentration of silica Ci,
ii) a step of calculating k₁, k₂, k_{B}, and kₐ in the three-step precipitation equilibrium reaction model of Formula (1), and calculating the predicted value of the concentration of silica dissolved C₁ from the initial concentration of silica Cᵢ, and k₁, k₂, kB, and ka, and
iii) a step of obtaining the predictive curve of the concentration of silica dissolved C₁ by plotting the concentration of silica dissolved C₁ versus the time t and fitting the curve based on the plotting results.

The step i) is a step of acquiring the initial concentration of silica Cᵢ. It can be assumed that the initial concentration of silica Cᵢ is equal to the total concentration of silica Cₜ in the calculation. Therefore, the initial concentration of silica Cᵢ can be obtained by the method described above, which is the same as in the total concentration of silica Cₜ.

In the step ii), k₁, k₂, k_{B}, and kₐ are calculated according to the flow chart of FIG. 1. The predicted value of the concentration of silica dissolved C₁ is calculated from the initial concentration of silica Cᵢ, and k₁, k₂, k_{B}, and kₐ. The predicted value of the concentration of silica dissolved C₁ is preferably acquired at different times, t, and can be calculated, for example, at 10 or more different time points, preferably at 50 or more different time points, and further preferably at 100 or more different time points. The predicted value of the dissolved concentration C₁ from the reaction start to a desired time point can be obtained thereby.

In the step iii), the concentration of silica dissolved C₁ versus the time t is plotted, and the predictive curve of the concentration of silica dissolved C₁ is obtained based on the plotting results. FIG. 3 is an example of the predictive curve of the concentration of silica dissolved C₁ obtained by the step iii), and is a predictive curve at a temperature of 100° C. and a pH of 7 obtained when the initial concentration is defined as about 1100 ppm. In FIG. 3, the solid line represents the concentration of silica dissolved C₁ (unit: ppm), and the alternate long and short dash line represents the amount of silica deposited (unit: ppm). The amount of silica deposited refers to the mass of a silica tetramer generated from the fluid per unit volume (1 L). When the fluid is geothermal water, the mass of the silica tetramer generated from the fluid per unit volume can be approximated to the mass of the silica tetramer generated from the fluid per unit mass (1 kg). Therefore, the sum of the concentration of silica dissolved C₁ and the amount of silica deposited is the initial concentration of silica Cᵢ.

The frequency factor A is necessary for fitting the polymerization temperature T and the predicted value of the concentration of silica dissolved C 1 in the initial stage of the reaction. Here, the initial stage of the reaction is also different depending on the reaction apparatus, the conditions, and the like, but refers to a stage at around 5 to 10 min after the reaction start. The relationship between the polymerization temperature T and the frequency factor A is represented by following Equation (3).

A=m[exp(nT)] (3)

In Equation (3), m and n are constants, and are values obtained by calculation described in the above-mentioned flow chart. T (K) represents the polymerization reaction temperature. The temperature range of T is about 250 K to 500 K. More specifically, m may be 2.0 to 3.1, and n may be 0.083 to 0.085. Preferably, m may be 2.3 to 2.8, and n may be 0.0835 to

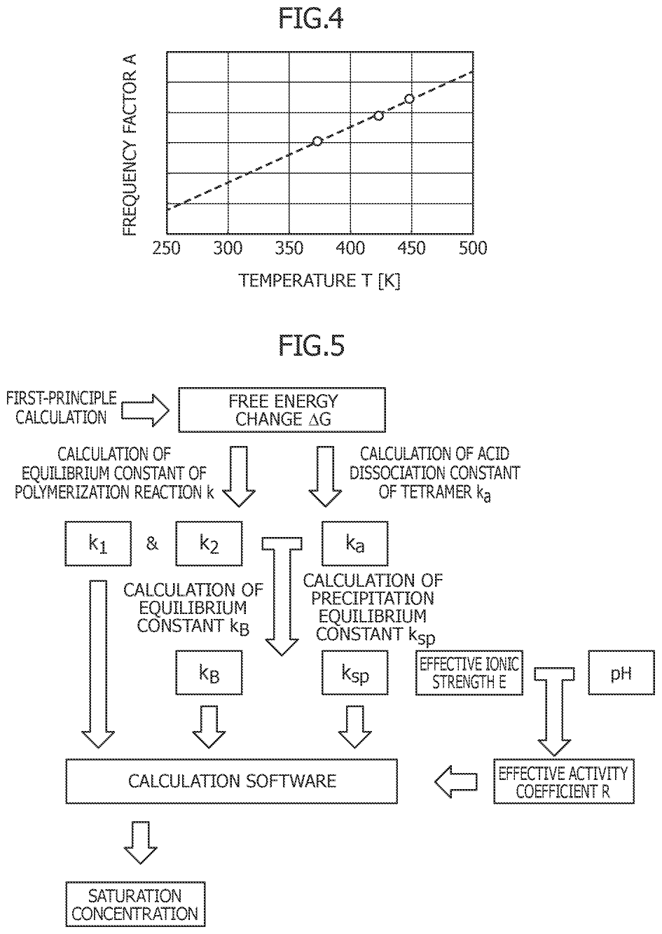

FIG. 4 is a semilogarithmic graph showing the predictive curve of the frequency factor A, and the vertical axis is a logarithmic scale. The use of the frequency factor A enables reproducing experimental values at temperatures at the initial stage of the reaction, for example at a time point of 5 to 10 min.

A specific method for predicting the amount of silica scale adhered based on the predictive curve of the concentration of silica dissolved C₁ will be described. The prediction method includes the following step A) to D):
A) a step of acquiring the time tₛ (min) until fluid containing silicic acid reaches the prediction portion,
B) a step of obtaining the concentration of silica dissolved C at the prediction portion from the time tₛ and the predictive curve of the concentration of silica dissolved C₁.
C) a step of acquiring the total concentration of silica Cₜ in fluid containing silicic acid, and
D) a step of calculating the amount of silica adhered at the time t based on the total concentration of silica Cₜ and the concentration of silica dissolved C₁.

In the step A), the time tₛ (min) until the fluid containing silicic acid reaches the prediction portion at which the adherence of silica scale needs to be predicted can be computed from the flow velocity of the fluid containing silicic acid in the plant system and the distance from the portion at which the time t=0 to the prediction portion. Alternatively, the time tₛ can be acquired by simulation based on plant system operation conditions. For example, in a geothermal power generation system, the time tₛ (min) may be time required for geothermal water drawn from a production well to reach a predetermined prediction portion.

The step B) can be implemented with an arithmetic unit that can perform an operation for calculating the concentration of silica dissolved C₁ from the predictive curve.

The steps C) and D) can be performed in the same way as the above-mentioned step c) and d). However, the total concentration of silica Cₜ according to the present aspect is equal to the initial concentration of silica Cᵢ. Accordingly, the initial concentration of silica Cᵢ used for deriving the concentration of silica dissolved C₁ can be calculated as the total concentration of silica Cₜ.

As mentioned above, according to the method for predicting the amount of silica scale adhered in accordance with the first aspect of the present embodiment, the saturation concentration of silica Ce₁ at a desired portion and/or the concentration of silica dissolved C₁ can be predicted without values taken from experience such as experimental values or observed values, and the prediction of the generated amount of silica scale that enables coping with complicated conditions in a short period of time at low estimated cost can be performed.

### Second Aspect

In a second aspect of the method according to the present embodiment, the generated amount of silica scale at any portion of a plant system is then predicted based on the predictive equation of the saturation concentration Ce₂ of the silica depending on the temperature T and the pH. In the second aspect, if the pH of the fluid dissolving silicic acid is 0 or more and less than 7, a particularly useful predictive equation is provided. Accordingly, the predictive equation according to the present aspect may also be referred to as a predictive equation in the acidic region. The saturation concentration of silica Ce₂ that the predictive equation of the present embodiment gives refers to the weight percent concentration (unit: ppm) of a solution in which silica that can be generated by the condensation polymerization reaction of silicic acid described above is dissolved, and the solution reaches a saturated solution at a predetermined temperature T and a predetermined pH. In other words, the saturation concentration of silica Ce₂ refers to the maximum concentration at which silica can be dissolved at the predetermined temperature T and the predetermined pH. The definition of silicic acid, silica, and silica scale is the same as in the first aspect.

FIG. 5 is a flow chart for calculating the saturation concentration of silica Ce₂ and the concentration of silica dissolved C₂. In the predictive equation in the acidic region, a calculation equation is also derived based on the three-step precipitation equilibrium reaction model of the silica polymerization represented by Formula (1), described above. The equation for computing silica acid dissociation constant kₐ: pkₐ=pΔG+q and preferable values of the constants p and q are also the same as in the first aspect.

The saturation concentration in the acidic region according to the second aspect Ce₂ is calculated based on the relationship between the polymerization reaction temperature T and the effective activity coefficient R as represented by the following Equation (4):

Ce₂=R{a₂[exp(b₂T)]} (4)

wherein, in Equation (4),
a₂ and b₂ are constants, and are values obtained by calculation described in the flow chart based on k₁, k₂, k_{B}, and kₐ. T (K) represents the polymerization reaction temperature. R is the effective activity coefficient, and is a value calculated based on pH.

For example, a₂ may be 16 to 36, and b₂ may be 0.003 to 0.015 based on specific calculation results. Preferably, a₂ may be 18 to 35, and b₂ may be 0.005 to 0.012. The most preferably, a₂ may be 20 to 33, and b₂ may be 0.006 to 0.010. The temperature range of T may be about 250 K to 500 K.

R can be computed based on the following calculation equation, and it is a coefficient that determines the influence of the pH in the acidic region (at a pH of 0 or more and less than 7) on the saturation concentration of silica.

−logR=A_{R}Z²{E/(1+B_{R}cE)} (5)

wherein, in Equation (5),
A_{R} and B_{R} are values calculated from the temperature of the silica polymerization reaction system T and the dielectric constant of water that is a solvent for polymerization reaction of silica ε based on the Debye-Huckel theory,
the charge number Z is 1 or 2, and the effective diameter coefficient c is 4, and
E is an effective ionic strength represented by the following Equation (6) and represented by the following equation:

—E={I+(hydrogen ion concentration)}/[1+B_{R}c[I+(hydrogen ion concentration)] (6)

wherein, in Equation (6), I is the solute ionic strength.
A_{R} and B_{R} can be more specifically represented by the following equations.

A_{R}=1.825*10⁶(εT)^{-3/2}

B_{R}=50.3*(εT)^{-1/2}
ε represents the dielectric constant of water at a temperature T, and T (K) represents the polymerization reaction temperature.

The charge number Z is 1 or 2. In the case of monovalent ions that are a silica monomer and a silica dimer (SiO(OH)³⁻ and Si₂O(OH)⁷⁻), the charge number Z is 1. In the case of divalent ions that are a silica monomer and a silica dimer (SiO₂(OH)₂²⁻ and SᵢO₂(OH)₆²⁻), the charge number Z is 2. The effective diameter coefficient c is a constant, that is 4, in the polymerization reaction of silica.

The solute ionic strength I is determined from the following equation:

I=1/2*(Cₜ+(hydrogen ion concentration))*Z²

In the equation, Cₜ is the total concentration of silica (unit: mol/L), and Z represents the charge number of the solute, and is 1 or 2.

The hydrogen ion concentration can be calculated from a predetermined pH of 0 or more and less than 7. For example, if the pH is 5.5, the hydrogen ion concentration is 10\hat{ }(−5.5).

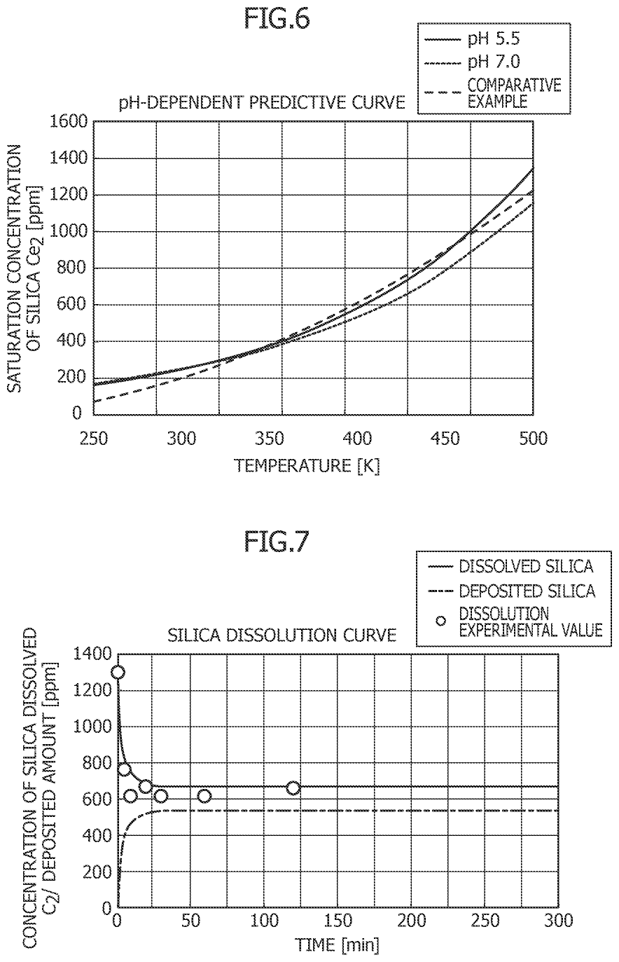

FIG. 6 is an example of Equation (4) representing the predictive curve of the saturation concentration of silica Ce₂. FIG. 6 shows a predictive curve at a pH of 5.5 and a predictive curve at a pH of 7.0 (in the first aspect) based on the predictive equation according to the second aspect of the present invention. As shown in FIG. 6, different predictive curves are obtained at different pHs according to the second aspect. Although the predictive curves are not shown, the predictive equations according to the second aspect can be derived by the above-mentioned method, and the predictive curves can be drawn at pHs in the range of 0 or more and less than 7. In addition, the predictive curve of Comparative Example was obtained based on the empirical rules by the method disclosed in Non-Patent Document 1, but the pH is not strictly considered, and the predictive curve is different from the predictive curve of the present aspect.

The dependence of the saturation concentration of silica Cee on the temperature T and the pH can be computed by the predictive equation according to the second aspect. The temperature T is the polymerization reaction temperature (unit: K) in the three-step precipitation equilibrium reaction model represented by Formula (1). When the amount of silica scale adhered is predicted in the prediction method according to the second aspect, the temperature at the predetermined prediction portion at which the amount of scale adhered needs to be predicted in the actual plant system Tₛ is used as the polymerization temperature, and the effective activity coefficient R is computed using the pH at the prediction portion and is used for predictive calculation. The temperature at the prediction portion Tₛ can be determined in the same way as in the first aspect. The pH at the prediction portion may be the pH of the fluid that can contain silica at the prediction portion in the plant system.

A specific method for predicting the amount of silica scale adhered based on Equation (4) will now be described. The prediction method includes the following steps a) to d):
a) a step of acquiring the temperature Tₛ (K) and the pH at the prediction portion at which the adherence of silica scale needs to be predicted,
b) a step of calculating the saturation concentration of silica Ce₂ at the prediction portion from the temperature Tₛ, the pH, and the predictive Equation (4),
c) a step of acquiring the total concentration of silica Cₜ in fluid containing silicic acid, and
d) a step of calculating the amount of silica adhered based on the total concentration of silica Cₜ and the saturation concentration of silica Ce₂.

The method for predicting the amount of silica scale adhered can be implemented in the same way as in the first aspect except that the pH at the prediction portion is acquired in the step a), and calculation is performed using the predictive Equation (4) in the step b). The value of the pH at the prediction portion can also be measured using a normal pH meter, and can be calculated and obtained by a method such as simulation.

According to the method for predicting the amount of silica scale adhered in accordance with the present embodiment, the adhered amount can be predicted under acidic pH conditions of the fluid under which silica scale is regarded as a problem. The adhered amount can be predicted more accurately than by conventional technology.

In the second aspect, the generated amount of silica scale at any portion of the plant system is also then predicted based on the predictive curve of the concentration of silica dissolved C₂ depending on the time. In the prediction method, the predictive curve showing the relationship between the time t and the concentration of silica dissolved C₂ are obtained by calculation, and the amount of silica adhered is calculated from the predictive curve. The method for acquiring the predictive curve of the concentration of silica dissolved C₂ depending on the time includes the following steps i) to iii):
i) a step of acquiring the initial concentration of silica Cᵢ,
ii) a step of calculating k₁, k₂, k_{B}, and kₐ in the three-step precipitation equilibrium reaction model in Formula (1), calculating the effective activity coefficient R, and calculating the predicted value of the concentration of silica dissolved C₂ from the initial concentration of silica Cᵢ, k₁, k₂, k_{B}, kₐ, and R, and
iii) a step of obtaining the predictive curve of the concentration of silica dissolved C₂ by plotting the concentration of silica dissolved C₂ versus the time t and fitting the curve based on the plotted results.

In the second aspect, the method for obtaining the predictive curve of the concentration of silica dissolved C₂ may be the same as in the first aspect, except that the effective activity coefficient R of the predictive Equation (4) needs to be calculated in view of the pH condition when the predicted value of concentration of silica dissolved C₂ is calculated in the step ii). The method for computing the frequency factor A and the preferable values of the constants m and n determining the frequency factor can be the same as in the first aspect.

FIG. 7 is an example of the predictive curve of the concentration of silica dissolved C₂ obtained by the steps i) to iii) of the second aspect, and is a predictive curve at a temperature of 150° C. and pH 5.5 obtained when the initial concentration is around 1300 ppm. In FIG. 7, the solid line represents the concentration of silica dissolved C (unit: ppm), and the alternate long and short dash line represents the amount of silica deposited (unit: ppm).

A specific method for predicting the amount of silica scale adhered based on the predictive curve of the concentration of silica dissolved C₂ will be described. The prediction method may be the same as in the steps A) to D) of the first aspect except that calculation is performed using the predictive curve of the concentration of silica dissolved C₂.

As mentioned above, according to the method for predicting the amount of silica scale adhered in accordance with the second aspect of the present embodiment, the saturation concentration of silica Cee and/or the concentration of silica dissolved C₂ depending on the temperature and the pH at a desired portion can be predicted, without any experience values, such as experimental values and observed values. The prediction of the generated amount of silica scale that enables coping with complicated conditions in a short period of time at low estimated cost can be performed. Specifically, the generated amount of silica scale in view of the polymerization reaction of silica at a pH in the acidic region can be predicted.

Third Aspect

In the third aspect of the method according to the present embodiment, the generated amount of silica scale at any portion of the plant system is then predicted based on the predictive equation of the saturation concentration Ce₃ of silica depending on the temperature T and the pH. In the third aspect, if the pH of fluid dissolving silicic acid is more than 7 and 14 or less, a particularly useful predictive equation is provided. Accordingly, the predictive equation according to the present aspect is also referred to as a predictive equation in the basic region. The definition of the saturation concentration of silica Ce₃ that the predictive equation of the third aspect gives is the same as in the second aspect, and the definition of silicic acid, silica, and silica scale is the same as in the first aspect.

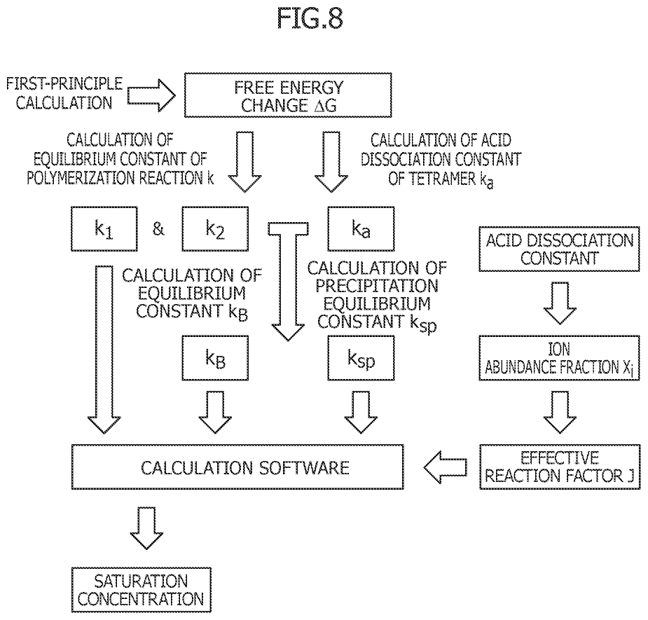

FIG. 8 is a flow chart for calculating the saturation concentration of silica Ce₃ and the concentration of silica dissolved C₃. In the predictive equation in the basic region, the calculation equation is also derived based on the three-step precipitation equilibrium reaction model of the silica polymerization represented by the above-mentioned Formula (1). The equation for computing the acid dissociation constant kₐ, pkₐ=pΔG+q, is also the same as in the first aspect, and the preferable values of the constants p and q are also the same.

The saturation concentration Ce₃ in the basic region according to the third aspect is calculated as represented by the following Equation (7) based on the relationship between the polymerization reaction temperature T and the effective reaction factor J.

Ce₃=(1−J)[a₃{exp(b₃T)}] (7)

wherein, in Equation (7),

a₃ and b₃ are constants, and are values obtained based on k₁, k₂, k_{B}, and kₐ by the calculation described in the flow chart. T (K) represents the polymerization reaction temperature. J is an effective reaction factor, and it is a value calculated based on the abundance fractions of the silica monomer ion and the silica dimer ion.

For example, a₃ may be 6 to 34, and b₃ may be 0.005 to 0.015 based on the specific calculation results. Preferably, a₃ may be 8 to 32, and b₃ may be 0.005 to 0.015. The most preferably, a₃ may be 10 to 30, and b₃ may be 0.006 to 0.009, and the temperature range of T may be around 250 K to 500 K.

J can be computed based on the following calculation equation, and is a coefficient determining the influence of the pH in the basic region on the saturation concentration of silica. This is based on the findings of the present inventors that ions do not participate in the polymerization reaction of silica directly in the basic region, and J is determined depending on the abundance fractions of molecular silica, which is not ionized. The effective reaction factor J can be represented by the following Equation (8).

J=(X−Xi₁−Xi₂)/X (8)

wherein X is the total amount of silica (molar amount: 100%), and Xi₁ and Xi₂ are abundance fractions of the silica monomer ion and the silica dimer ion (molar fraction %), and can calculated from the acid dissociation constant kₐ. The silica monomer ion refers to Si(OH)₃O⁻, and the silica dimer ion refers to Si₂(OH)₇O⁻. The acid dissociation constant kₐⱼ is an equilibrium constant in view of dissociative reaction in which protons (hydrogen ions) are released from silica monomer, silica dimer, and silica tetramer molecules.

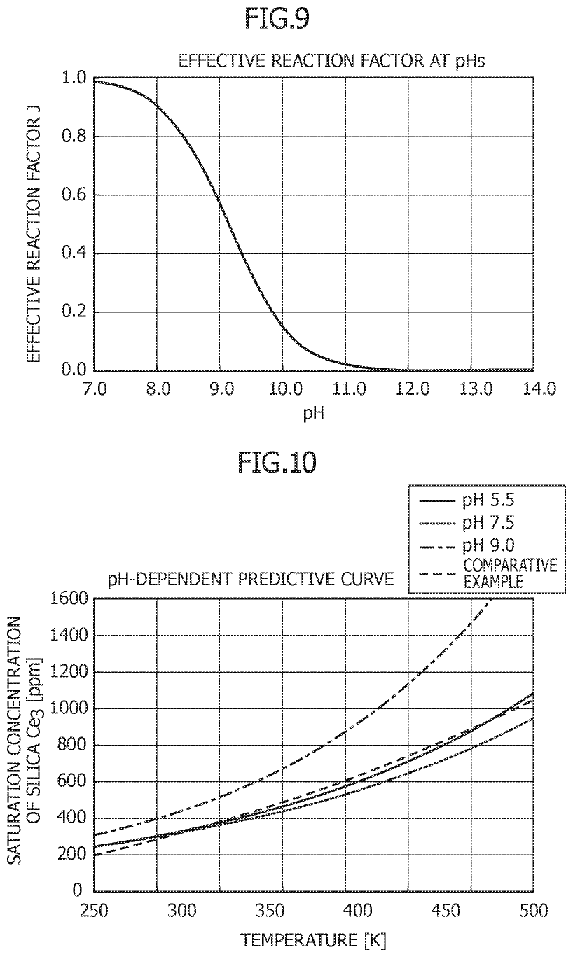

J is a number between 0 and 1, and the pH changes between 7 and 14. FIG. 9 shows a graph of a change in J versus the pH. The effective reaction factor J at a specific pH in the basic regional can be obtained based on the graph in FIG. 9.

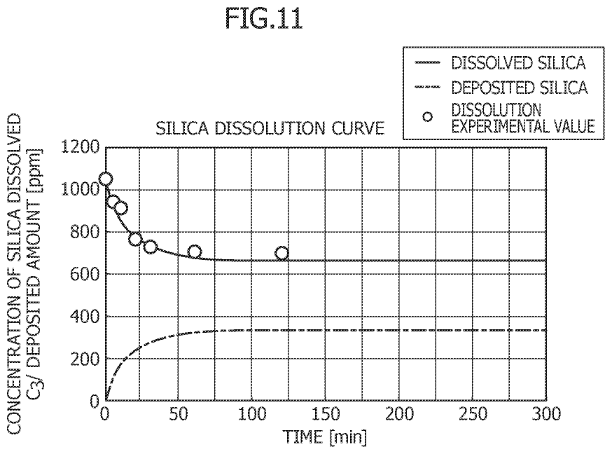

FIG. 10 is an example of Equation (7) represented by the predictive curve of the saturation concentration of silica Ce₃. The predictive curve at pH 9.0 and the predictive curve at pH 7.0 (in the first aspect) based on the predictive equation according to the third aspect of the present invention are shown. Although predictive curves are not shown, the predictive equations according to the third aspect can be derived by the above-mentioned method at pHs in the range of more than pH 7 and 14, and the predictive curves can be drawn. The predictive curve at a pH of 5.5 based on the predictive equation according to the second aspect is also drawn together in the graph. As shown in FIG. 10, according to the third aspect, different predictive curves are obtained at different pHs. In addition, the predictive curve of the Comparative Example was obtained based on empirical rules by the method disclosed in Non-Patent Document 1, but the pH is not strictly considered, and the predictive curve is different from the predictive curve of the present aspect.

The dependence of the saturation concentration of silica Ce₃ on the temperature T and the pH can be computed by the predictive equation according to the third aspect. The temperature T is the polymerization reaction temperature (unit: K) in the three-step precipitation equilibrium reaction model represented by Formula (1). In the prediction method according to the third aspect, the steps of predicting the amount of silica scale adhered is the same as in the second aspect except that the saturation concentration of silica Ce₃ is calculated based on Equation (7). According to the method for predicting the amount of silica scale adhered in accordance with the third aspect, the adhered amount can be predicted under basic pH conditions of the fluid under which silica scale is regarded as a problem, and the amount of silica scale adhered specifically in the basic fluid can be accurately predicted.

In the third aspect, the generated amount of silica scale at any portion of the plant system can be also predicted based on the predictive curve of the concentration of silica dissolved C₃ depending on the time in the same way as in the second aspect. In the prediction method, the predictive curve showing the relationship between the time t and the concentration of silica dissolved C₃ is obtained by calculation, and the amount of silica adhered is calculated from the predictive curve. The steps of the method for acquiring the predictive curve of the concentration of silica dissolved C₃ depending on the time are the same as in the second aspect except that k₁, k₂, k_{B}, and kₐ are calculated in the three-step precipitation equilibrium reaction model of Formula (1) in step ii) of the second aspect, the effective reaction factor J is calculated, and the predicted value of the concentration of silica dissolved C₃ is calculated from the initial concentration of silica Ci and k₁, k₂, k_{B}, kₐ, and J. That is, the steps may be the same as in the first aspect except that when the predicted value of the concentration of silica dissolved C₃ is calculated, the effective reaction factor J in predictive Equation (7) needs to be calculated in view of the basic pH condition. The method for computing the frequency factor A and the preferable values of the constants m and n determining the frequency factor can also be the same as in the first aspect.

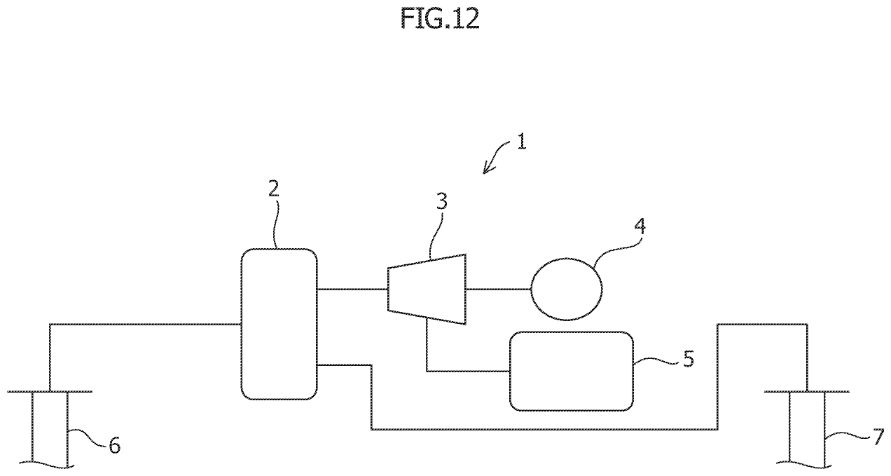

FIG. 11 is an example of the predictive curve of the concentration of silica dissolved C₃ obtained by the steps i) to iii) that are the same as in the second aspect, and is a predictive curve at a temperature of 100° C. and pH 9 obtained at an initial concentration of around 1050 ppm. In FIG. 11, the solid line represents the concentration of silica dissolved C₃ (unit: ppm), and the alternate long and short dash line represents the amount of silica deposited (unit: ppm).

As mentioned above, according to the method for predicting the amount of silica scale adhered in accordance with the third aspect of the present embodiment, the saturation concentration of silica Ce₃ and/or the concentration of silica dissolved C₃ depending on temperature and pH at a desired portion can be predicted without needing experience values such as experimental values or observed values, and the prediction of the generated amount of silica scale that enables coping with complicated conditions in a short period of time at low estimated cost can be performed. The generated amount of silica scale in view of the polymerization reaction of silica specifically at a pH in the basic region can be predicted.

### Second Embodiment: System for Predicting Amount of Silica Scale Generated

According to a second embodiment, the present invention is a system for predicting the amount of silica scale adhered, and includes the following:
a device that acquires a temperature at a prediction portion at which the adherence of silica scale needs to be predicted, Tₛ (K), and/or time until fluid containing silicic acid reaches the prediction portion, tₛ (min), and
a device that calculates the amount of silica adhered at the prediction portion based on the predictive equation of the saturation concentration of silica depending on the temperature Ce₁, Ce₂, or Ce₃ and/or the predictive curve of the concentration of silica dissolved depending on the time C₁, C₂, or C₃.

In the second embodiment, the predictive equation of the saturation concentration of silica Ce₁, Ce₂, or Ce₃ and the predictive curve of concentration of silica dissolved C₁, C₂, or C₃ versus the time can also be obtained in the same way as in the first aspect to the third aspect of the first embodiment, and description thereof is omitted. A device that acquires the temperature Tₛ (K) and/or the time tₛ (min), a device that calculates the amount of silica adhered, and a device for measuring the pH can also be selected from options that are the same as the options of the devices specifically mentioned for performing the steps in the aspects in the first embodiment.

The system for predicting the generated amount of silica scale according to the present embodiment can be incorporated into various plant systems, and the generated amount of silica scale can be predicted under the conditions of the plant systems.

### Third Embodiment: Geothermal Power Generation System

According to a third embodiment, the present invention relates to a geothermal power generation system, and comprises:
a gas-liquid separator that separates geothermal fluid drawn from a production well into a gas component and a liquid component,
a turbine that is disposed downstream of the gas-liquid separator and that is configured to be rotatable by the gas component separated in the gas-liquid separator,
piping that delivers the liquid component separated in the gas-liquid separator to an injection well, and
the system for predicting a generated amount of silica scale of the second embodiment.

FIG. 12 is a conceptual diagram illustrating a geothermal power generation system according to the third embodiment. With reference to FIG. 12, a geothermal power generation system 1 mainly comprises a production well 6, a gas-liquid separator 2, a turbine 3, a generator 4, a condenser 5, an injection well 7, and a system for predicting the generated amount of silica scale. The system for predicting the generated amount of silica scale is a system described in the second embodiment, and is the system that can implement the prediction method described in the first embodiment.

The flow of substances in the geothermal power generation system 1 will be described. The production well 6 is a well that guides out hot water, steam, or a mixture thereof (hereinafter referred to as geothermal fluid) present in the underground geothermal reservoir. The geothermal fluid guided out of the production well 6 is separated into steam, which is a gas component, and hot water, which is a liquid component, in the gas-liquid separator 2. The separated steam is guided to the turbine 3, and it is used for rotating the turbine 3 to generate electricity in the generator 4. The steam that passes the turbine 3 is cooled in the condenser 5 and guided to the injection well 7 through piping (not shown). In addition, the hot water separated in the gas-liquid separator 2 is cooled and guided to the injection well 7. The geothermal power generation system can include an aspect in which the steam separated in the gas-liquid separator 2 rotates the turbine directly, and an aspect in which the steam heats a solvent having a low boiling point, and the solvent having a low boiling point rotates the turbine. In the present invention, the “turbine configured to be rotatable by the gas component separated in the gas-liquid separator” includes both aspects.

The prediction portion at which the adherence of silica scale needs to be predicted in the plant system is not particularly limited, and may be any point to which geothermal fluid can adhere. The prediction portion may be the turbine member and the like exemplified in the first embodiment, but is not limited to these. The prediction portion may be a member that is not specifically shown in FIG. 12. The prediction portion may be one point and two or more points in the plant system, and the upper limit of the number of the prediction portions is not limited theoretically.

According to the geothermal power generation system in accordance with the present embodiment, the amount of silica scale adhered can be accurately predicted, maintenance can be performed at suitable times with the number of the operation stoppages of the system minimized, and power can therefore be generated steadily and highly efficiently. Specifically, the amount of silica scale adhered can be predicted also in view of the pH of the geothermal fluid flowing through the geothermal power generation system.

### EXAMPLES

Hereinafter, the present invention will be described in more detail by giving Examples of the present invention. However, the present invention is not limited to the scope of the following Examples.

A model to handle the silica polymerization reaction was made with a reaction module of calculation software (COMSOL Multiphysics® modeling software) using Formula (1) as a reaction model of silica polymerization reaction in hot water.

(1) Calculation Results by First Aspect

ΔGₙ of the silica polymerization reaction at 25° C., 100° C., 150° C., and 175° C. were calculated using the density functional theory of first-principle calculation. The n represented the order of the polymerization reaction, and ΔGₙ were calculated in the cases of the first step that was the polymerization reaction of the monomer (n=1) and the second step that was the polymerization reaction of the dimer (n=2). The equilibrium constants k₁ and k₂ were computed from ΔGₙ based on the thermodynamic definition using the following Formulae:

$${k}_{1} = exp ( - \frac{Δ {G}_{1}}{R T} ) \\ {k}_{2} = exp ( - \frac{Δ {G}_{2}}{R T} ) \qquad [ Formula 4 ]$$

Table 1 shows the results of calculating pKa from the computed ΔGₙ of the acid dissociation reaction of silica using the predictive equation of the acid dissociation constant of silica that the applicant established and computing Ka further using a general equation for defining the acid dissociation constant in comparison with the Comparative Example, which is a conventional technique, and an experimental value.

TABLE 1

| Subject | Calculation | | Comparative | Experimental |
|---|---|---|---|---|
| molecule | item | Example | Example | value |
| Si(OH)₄ | ka | 1.49E−04 | 1.89E−38 | 5.22E−05 |
| | pka | 8.81 | 37.72 | 9.86 |

In the acid dissociation reaction shown as follows:

HA⇄H⁺+A⁻ [Formula 5]
wherein HA is a general formula of an acid,
the acid dissociation constant is incidentally calculated based on the definition of the acid dissociation constant as follows:

$$Δ G = G ( {A}^{-} ) + G ( {H}^{+} ) - G ( H A ) {p K}_{a} = \frac{Δ G}{( ln 10 ) R T} \qquad [ Formula 6 ]$$

Since a proton did not have an electron, G(H⁺) was not able to be calculated. In the Comparative Example, G(H₃O⁺) was therefore substituted approximately, and ΔG was calculated. In addition, the experimental value is the literature value of the acid dissociation constant of the monomer.

When a numerical equation was derived from the chemical reaction model, the ionization equilibrium constant k_{B} and the precipitation equilibrium constant kₛₚ, could be calculated as follows:

$${k}_{B} = \frac{{k}_{2}}{{k}_{a}} {k}_{s p} \approx {k}_{2} \times {k}_{a} \qquad [ Formula 7 ]$$

The above-mentioned various parameters and frequency factor A were substituted in the software, and the predictive curve of the concentration of silica dissolved C₁ was found. A point at which the concentration of silica dissolved was in equilibrium was estimated as the saturation concentration from the obtained predictive curve of the concentration of silica dissolved C₁.

FIG. 3 shows an example of the predictive curve of the concentration of silica dissolved C₁ at T=373.15 K. Here, the dissolution experimental value refers to the saturation concentration of the silica according to the results of a hydrothermal synthesis experiment that simulated fluid in power generation facilities using a small-scale experimental apparatus. The Comparative Example shows the saturation concentration of silica predicted by a conventional technique. Since, in the conventional technique, the time-dependent calculation results could not be obtained, only one point was plotted.

The obtained saturation curve was subjected to variance analysis, and the predictive equation of the saturation concentration of silica was established. The predictive equation is represented by the above-mentioned Equation (2). The established predictive equation of the saturation concentration was inspected by comparing the predicted value of the saturation concentration (Example) with the calculation results of the Comparative Example described above and the experimental values. In FIG. 2, the solid line is the calculation results of the Example, the broken line is the calculation results of the Comparative Example, and the white circles are experimental values. FIG. 2 shows that the predicted values of the Example according to the present technique matched the experimental values closely, and accurate prediction was able to be performed.

A method for correcting the frequency factor A, which was necessary for fitting the reaction initial concentration, was devised, and the variance analysis was performed. The frequency factors A at pH 7 under the temperature conditions of 373.15 K, 423.15 K, and 448.15 K were plotted. FIG. 4 shows the results. Equation (3) was obtained from FIG. 4, and an equation showing the temperature dependence of the frequency factor A was established.

(2) Calculation Results by Second Aspect

In the second aspect, calculation was performed using the values of k₁, k₂, k_{B}, and kₐ, which were the same as in the first aspect, and further using the effective activity coefficient R. If T=373.15 [K], parameters for calculating R were: ε=55.72 [F/m], Cₜ=1103.07 [ppm], I=0.006 [mol/L], A_{R}=0.609, and B_{R}=0.349, and R was computed as 0.908. If T=423.15 [K], the parameters were: ε=44.24 [F/m], C=1335.43 [ppm], I=0.007 [mol/L], A_{R}=0.713, and B_{R}=0.368, and R was computed as 0.885. If T=448.15 [K], the parameters were: ε=39.20 [F/m], C=1474.43 [ppm], I=0.008 [mol/L], A_{R}=0.784, and B_{R}=0.380, and R was calculated as 0.869.

FIG. 7 shows an example of the predictive curve of the concentration of silica dissolved C₂ at 150° C. (423.15 K) and pH 5.5. The dissolution experimental values are the same as in the dissolution experiment by the first aspect except that the experiment was performed at pH The obtained saturation curve was subjected to variance analysis, and the predictive equation of the saturation concentration of silica was established. The same value as in the first aspect was used as the frequency factor A. The predictive equation is represented by the above-mentioned Equation (4). It was shown that the plotting of the dissolution experimental values matched the predicted values at pH 5.5 by the present technique closely, and accurate prediction was able to be performed.

(3) Calculation Results by Third Aspect

In the third aspect, calculation was performed using the values of k₁, k₂, k_{B}, and kₐ, which were the same as in the first aspect, and further using the effective reaction factor J. Parameters used for deriving the effective reaction factor J were: Xi1=15.20% by mol, Xi2=27.35% by mol, X=100, and J was finally computed as 0.575.

FIG. 11 shows an example of the predictive curve of the concentration of silica dissolved C₃ at 100° C. (373.15 K) and pH 9.0. The dissolution experimental values are the same as in the dissolution experiment by the first aspect except that the experiment was performed at a pH of 9.0. The obtained saturation curve was subjected to variance analysis, and the predictive equation of the saturation concentration of silica was established. The same value as in the first aspect was used as the frequency factor A. The predictive equation is represented by the above-mentioned Equation (7). It was shown that the plotting of the dissolution experimental values matched the predicted values at pH 9.0 by the present technique closely, accurate prediction could be performed.

### REFERENCE SYMBOL LIST

1 Geothermal power generation plant
2 Gas-liquid separator
3 Turbine
4 Generator
5 Condenser
6 Production well
7 Injection well

The invention claimed is:

1. A method for predicting a generated amount of silica scale, comprising steps of:
acquiring a temperature at a prediction portion at which adherence of silica scale needs to be predicted, Tₛ (K), and/or time until fluid containing silicic acid reaches the prediction portion, tₛ (min), and
calculating an amount of silica adhered at the prediction portion based on a predictive equation of a saturation concentration of silica depending on the temperature and/or a predictive curve of a concentration of silica dissolved depending on the time,
wherein the predictive equation of the saturation concentration of silica and the predictive curve of the concentration of silica dissolved are obtained based on k₁, k₂, k_{B}, and kₐ in a three-step precipitation equilibrium reaction model represented by the following Formula (1): 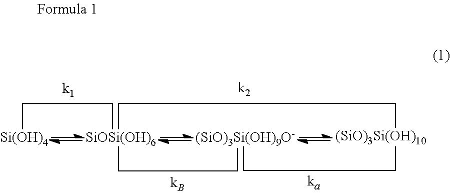
wherein
k₁ is a reaction equilibrium constant between Si(OH)₄ and SiOSi(OH)₆,
k₂ is a reaction equilibrium constant between SiOSi(OH)₆ and (SiO)₃OSi(OH)₁₀,
k_{B} is an ionization equilibrium constant between SiOSi(OH)₆ and (SiO)₃Si(OH)₉O⁻, and
kₐ is a silica acid dissociation constant between (SiO)₃Si(OH)₉O⁻ and (SiO)₃Si(OH)₁₀.

2. The method according to claim 1, wherein the silica acid dissociation constant kₐ is obtained by quantum chemical calculation and linear fitting correction based on free-energy change ΔG in equilibrium reaction between (SiO)₃Si(OH)₉O⁻ and (SiO)₃OSi(OH)₁₀.

3. The method according to claim 2, wherein the relationship between the silica acid dissociation constant kₐ and the free-energy change ΔG is represented by the following equation:
pkₐ=pΔG+q
wherein p and q are constants.

4. The method according to claim 3, wherein p is 0.19 to 0.24, and q is −56 to −51.

5. The method according to claim 1,
wherein the predictive curve of the concentration of silica dissolved C is obtained by fitting plotting of concentrations of silica dissolved at two or more different time points obtained by first-principle calculation based on an initial concentration of silica, Cᵢ, and Formula (1),
a frequency factor to be used for correcting the fitting in an initial stage of the reaction, A, is represented by the following equation:
A=m[exp(nT)] (3)
wherein m and n are constants, and are calculated based on k₁, k₂, k_{B}, and kₐ.

6. The method according to claim 5, wherein m is 2.0 to 3.1, and n is 0.083 to 0.085.

7. The method according to claim 1, wherein the predictive equation of the saturation concentration of silica Ce is represented by a saturation concentration of silica at a temperature T, Ce₁:
Ce₁=a₁[exp(b₁T)] (2)
wherein
a₁ and b₁ are constants calculated based on k₁, k₂, k_{B}, and kₐ, and
T represents polymerization reaction temperature.

8. The method according to claim 7, wherein a₁ is 18 to 32, and b₁ is 0.005 to 0.010.

9. The method according to claim 1, wherein a predictive equation of the saturation concentration of silica Ce is represented by a saturation concentration of silica at a temperature T and a pH of 0 or more and less than 7, Ce₂:
Ce₂=R{a₂[exp(b₂T)]} (4)
wherein
a₂ and b₂ are constants calculated based on k₁, k₂, k_{B}, and kₐ,
R is an effective activity coefficient calculated based on the pH, and
T represents polymerization reaction temperature.

10. The method according to claim 9, wherein a calculation equation of the effective activity coefficient R is represented by the following equation:
−logR=A_{R}Z²{E/(1+B_{R}cE)} (5)
wherein
A_{R}=1.825*10⁶(εT)^{-3/2},
B_{R}=50.3*(εT)^{-1/2},
a charge number, Z, is a constant selected from 1 or 2, and an effective diameter coefficient, c, is 4,
E is an effective ionic strength represented by the following Equation (6):
E={I+(hydrogen ion concentration)}/[1+B_{R}c[I+(hydrogen ion concentration)] (6)
wherein I is a solute ionic strength.

11. The method according to claim 9, wherein a₂ is 16 to 36, and b₂ is 0.003 to 0.015.

12. The method according to claim 1, wherein a predictive equation of the saturation concentration of silica Ce₃ is represented by a saturation concentration of silica at a temperature T and a pH of more than 7 and 14 or less, Ce₃:
Ce₃=(1−J){a₃[exp(b₃T)]} (7)
wherein
a₃ and b₃ are constants calculated based on k₁, k₂, k_{B}, and kₐ,
J is an effective reaction factor calculated based on abundance fractions of a silica monomer ion and a silica dimer ion, and
T represents polymerization reaction temperature.

13. The method according to claim 12, wherein a calculation equation of the effective reaction factor J is represented by the following equation:
J=(X−Xi₁−Xi₂)/X (8)
wherein
X is a total amount of silica,
Xi₁ is the abundance fraction of the silica monomer ion calculated from an acid dissociation constant, kₐⱼ, and
Xi₂ is the abundance fraction of the silica dimer ion calculated from the acid dissociation constant kₐⱼ.

14. The method according to claim 12, wherein a₃ is 6 to 34, and b₃ is 0.005 to 0.015.

15. The method according to claim 1, wherein the amount of silica adhered is predicted by
a step of acquiring a total concentration of silica in the fluid containing silicic acid, Cₜ, and
a step of calculating the amount of silica adhered based on the total concentration of silica Cₜ and the saturation concentration of silica.

16. The method according to claim 1, wherein the amount of silica adhered is predicted by a step of calculating the amount of silica adhered based on the predictive curve of the concentration of silica dissolved.

17. A system for predicting a generated amount of silica scale, comprising:
a device that acquires a temperature at a prediction portion at which adherence of silica scale needs to be predicted, Tₛ (K), and/or time until fluid containing silicic acid reaches the prediction portion, tₛ (min), and
a device that calculates an amount of silica adhered at the prediction portion based on a predictive equation of a saturation concentration of silica depending on the temperature and/or a predictive curve of a concentration of silica dissolved depending on the time,
wherein the predictive equation of the saturation concentration of silica and the predictive curve of the concentration of silica dissolved are obtained based on k₁, k₂, k_{B}, and kₐ in a three-step precipitation equilibrium reaction model represented by the following Formula (1): 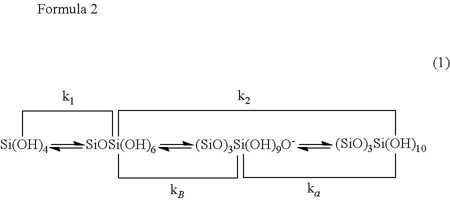
wherein
k₁ is a reaction equilibrium constant between Si(OH)₄ and SiOSi(OH)₆,
k₂ is a reaction equilibrium constant between SiOSi(OH)₆ and (SiO)₃OSi(OH)₁₀,
k_{B} is an ionization equilibrium constant between SiOSi(OH)₆ and (SiO)₃Si(OH)₉O⁻, and
kₐ is a silica acid dissociation constant between (SiO)₃Si(OH)₉O⁻ and (SiO)₃OSi(OH)₁₀.

18. A geothermal power generation system, comprising:
a gas-liquid separator that separates geothermal fluid drawn from a production well into a gas component and a liquid component;
a turbine that is disposed downstream of the gas-liquid separator and that is configured to be rotatable by the gas component separated in the gas-liquid separator;
piping that delivers the liquid component separated in the gas-liquid separator to an injection well; and
the system for predicting a generated amount of silica scale according to claim 17.
